SEQUENCE LISTING (13 sequences)

SEQ ID NO: 1 - DNA, 25 bp, Artificial Sequence
  Oligonucleotide
ccgttacgccaccagctccaaacgg

SEQ ID NO: 2 - DNA, 25 bp, Artificial Sequence
  Oligonucleotide
ccgttacgccaccagctccaaacgg

SEQ ID NO: 3 - DNA, 25 bp, Artificial Sequence
  Oligonucleotide
ccgttacgccaccagctccaaacgg

SEQ ID NO: 4 - DNA, 17 bp, Artificial Sequence
  Oligonucleotide
ttggagctggtggcgta

SEQ ID NO: 5 - DNA, 25 bp, Artificial Sequence
  Oligonucleotide
ccgttacgccaccagctccaaacgg

SEQ ID NO: 6 - DNA, 25 bp, Artificial Sequence
  Oligonucleotide
ccgttcctccggttcatgccaacgg

SEQ ID NO: 7 - DNA, 25 bp, Artificial Sequence
  Oligonucleotide
ccgttacggccctgaagtacaacgg

SEQ ID NO: 8 - DNA, 17 bp, Artificial Sequence
  Oligonucleotide
ttggagctggtggcgta

SEQ ID NO: 9 - DNA, 17 bp, Artificial Sequence
  Oligonucleotide
tggcatgaaccggagga

SEQ ID NO: 10 - DNA, 17 bp, Artificial Sequence
  Oligonucleotide
tgtacttcagggccgta

SEQ ID NO: 11 - DNA, 28 bp, Artificial Sequence
  Oligonucleotide
ttcccgtcttccagacaagagtgcaggg

SEQ ID NO: 12 - DNA, 35 bp, Artificial Sequence
  Oligonucleotide
caggctacggcacgtagagcatcaccatgatcctg

SEQ ID NO: 13 - protein, 15 aa, Artificial Sequence
  Synthetically Generated Polypeptide
TINEEAAEWDRVHPK

(19) United States
(12) Patent Application Publication
(10) Pub. No.: US 2011/0210017 A1
(43) Pub. Date: Sep. 1, 2011

(54) FABRICATION OF ELECTROCHEMICAL BIOSENSORS VIA CLICK CHEMISTRY

(76) Inventors: Rebecca Y. Lai, Lincoln, NE (US); Socrates Canete, Dumaguete City (PH)

(21) Appl. No.: 13/037,825

(22) Filed: Mar. 1, 2011

Related U.S. Application Data
(60) Provisional application No. 61/309,152, filed on Mar. 1, 2010.

(51) Int. Cl. G01N 27/327; B23P 11/00; G01N 33/487; G01N 33/50

(52) U.S. Cl. 205/792; 29/428; 204/403.01; 204/403.02

(57) ABSTRACT

This disclosure describes electrochemical biosensors produced using click chemistry.

### CROSS REFERENCE TO RELATED APPLICATIONS

This application claims benefit under 35 U.S.C. 119(e) to U.S. Application No. 61/309,152, filed on Mar. 1, 2010.

### FEDERALLY SPONSORED RESEARCH OR DEVELOPMENT

This invention was made with government support under Grant No. DMR-0820521 awarded by National Science Foundation. The government has certain rights in the invention.

### TECHNICAL FIELD

This disclosure generally relates to electrochemical biosensors.

### BACKGROUND

The use of electrochemical sensors in the medical field for testing various blood or urine analytes and in the environmental field for monitoring water or soil contamination is well known. In the presence of a target analyte in a sample, a difference in potential is generated across two or more of the electrodes of the electrochemical sensor. Recently, an electrochemical sensor that is dependent upon conformational changes in a biopolymer has been developed that is fully electronic and requires neither optics nor high voltage power supplies. This disclosure describes methods of fabricating such electrochemical sensors that is extremely chemoselective and still results in a durable electrochemical biosensor.

### SUMMARY

This disclosure describes electrochemical biosensors produced using click chemistry.

In one aspect, an electrochemical biosensor is provided. Such a biosensor typically includes at least two electrodes and a binding ligand, wherein one end of the binding ligand is linked to at least one of the electrodes via a 1,2,3 triazole moiety, and wherein a second end of the binding ligand comprises a redox tag. Representative binding ligands include, without limitation, nucleic acids, aptamers, polypeptides, and proteins. In certain embodiments, the electrochemical biosensor is disposable. In certain embodiments, the electrochemical biosensor is reusable. In some instances,

one or more of the electrodes can be gold-plated. In one embodiment, the redox tag is methylene blue.

In another aspect, a method of making an electrochemical biosensor is provided. Such a method typically includes providing an electrochemical sensor comprising a working electrode, a reference electrode, and a counter electrode; and attaching a binding ligand to the substrate using click chemistry, wherein the binding ligand comprises a redox tag. Representative binding ligands include, without limitation, nucleic acids, aptamers, polypeptides, and proteins. In certain embodiments, the electrochemical biosensor is disposable. In certain embodiments, the electrochemical biosensor is reusable. In one embodiment, the redox tag is methylene blue.

In still another aspect, a method of detecting the presence or absence of a target analyte in a sample is provided. Such a method typically includes contacting an electrochemical biosensor as described herein with a sample; determining whether or not the electrochemical biosensor exhibits a change in redox potential. Generally, a change in the redox potential is indicative of the presence of the target analyte and little to no change in the redox potential is indicative of the absence of the target analyte. Representative samples include biological samples and environmental samples.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 shows AC voltammograms of E-DNA sensors fabricated via conventional “two step approach” (top) and click chemistry (bottom) before hybridization, after incubation with 1.0 μM of target WT-Gly DNA, and after regeneration via a simple, room temperature deionized water rinse. Conditions: AC frequency: 10 Hz; AC amplitude: 25 mV; incubation time: ˜50 min (top) and ˜60 min (bottom); medium: Phys2 buffer (pH 7.4).

FIG. 2 shows similar sensor reusability of E-DNA sensors fabricated via conventional “two-step approach” (top) and click chemistry (bottom).

FIG. 3 shows that E-DNA sensors fabricated via conventional “two-step approach” (top) and click chemistry (bottom) show similar hybridization kinetics.

FIG. 4 shows AC voltammograms of E-DNA sensors fabricated via conventional “two-step approach” (top) and click chemistry (bottom) before hybridization, after incubation with 1.0 μM of target WT-Gly DNA, and after regeneration via a 4-min room temperature incubation in 4 M GHCl. Conditions: AC frequency: 10 Hz; AC amplitude: 25 mV; incubation time: ˜60 min (top) and ˜50 min (bottom); medium: undiluted human male whole blood.

FIG. 5 shows a schematic of the conventional (top) and click chemistry-based (bottom) E-DNA sensor constructs.

FIG. 6 shows AC voltammograms of click chemistry-based E-DNA sensors fabricated in the absence of copper sulfate (top) or sodium ascorbate (bottom) before, after incubation with 1 μM of target DNA, and after regeneration via a 30-sec room temperature DI water rinse. Since sensor regeneration is a unique property of the E-DNA sensor, it can be used as a gauge for sensor performance. Sensors with physically-adsorbed probes cannot be regenerated since the probes will be desorbed by successive voltammetric scans.

FIG. 7 shows the E-DNA sensor response to human whole blood. A ˜37% signal reduction is evident when the sensor was transferred from a Phys2 buffer to the whole blood matrix. The reduction in current is likely due to the differences in the viscosity and ionic strength, which is known to affect the MB current in E-DNA sensors.

FIG. 8 shows a cyclic voltammograms of the Cu-TBTA complex obtained in a deacerated 1:1 DMSO/H₂O solution containing 150 mM TBTA, 75 mM CuSO₄ and 0.1 M NaCl, using a C11-OH/C11-N₃ SAM-modified gold electrode and a bare glassy carbon electrode.

FIG. 9 shows the equilibration-interrogation-regeneration cycles of the three DNA sensors in the array. Area A indicates the time used to reach sensor equilibration prior to target interrogation. Areas B, C and D indicate sensor interrogation with Brca2, p. 53 and K-ras target DNA, respectively. Area E indicates sensor regeneration. Inset: the microfabricated 3-pixel array used.

FIG. 10 shows a schematic of an E-DNA sensor that consists of a redox-labeled stem-loop DNA probe covalently attached to an interrogating electrode. The E-DNA signal arises due to the binding-induced change in the conformation of the stem-loop probe and the efficiency with which the attached redox label transfers electrons to the electrode. In the absence of target, the stem-loop structure holds the redox label in proximity to the electrode, enabling efficient electron transfer. Upon hybridization with the complementary target DNA, the double-stranded conformation of the probe DNA forces the redox label away from the electrode, impeding electron transfer and leading to a detectable reduction in redox current.

FIG. 11 shows a Sharpless click chemistry reaction. For a click chemistry-based E-DNA sensor: R=C3-DNA-C7-methylene blue; R′═HS—(CH₂)₁₁; and TBTA=tris[(1-benzyl-1H-1,2,3-triazol-4-yl)methyl]amine.

FIG. 12 shows the structures of the C7-MB probe (top) and the alkyne-modified DNA probe (bottom).

FIG. 13 shows AC voltammograms of C7-MB (dotted line) and K-ras probe-modified E-DNA sensor (solid line) fabricated via potential-assisted click chemistry under the same experimental condition. The voltammograms were recorded in a Phys2 buffer from −0.05 V to −0.52 V vs. Ag/AgCl with a 10 Hz, 25 mV AC potential.

FIG. 14 shows AC voltammograms obtained from the 3-pixel E-DNA sensor array. Shown are the sensors' responses to the added target DNA. The sensors were interrogated in sequence, in which the Brca2 target was first added (top). A large signal suppression was observed in Pixel 1, the pixel that was selectively modified with the Brca2 DNA probes. No signal suppression was observed with the two pixels that were modified with p53 and K-ras probes. The p53 target DNA was added once hybridization was complete for the Brca2-modified pixel (center). The K-ras target was the last to be added (bottom).

FIG. 15 shows the lowest energy structures of the Brca2 (top left), p. 53 (top right) and K-ras (bottom) DNA probes as predicted by m-Fold. The simulation was performed at 23° C. and in presence of 0.15 M Na⁺ and 0.002 M Mg²⁺. As observed, the K-ras probe has a mini-loop structure that is absent in the Brca2 and p. 53 probes. This structure affects target hybridization both kinetically and thermodynamically.

FIG. 16 shows an AC voltammogram obtained from an electrochemical aptamer-based (E-AB) sensor fabricated using click chemistry for detection of vascular endothelial growth factor (VEGF) in 50% blood serum.

FIG. 17 shows an AC voltammogram obtained from an electrochemical aptamer-based (E-AB) sensor fabricated via click chemistry for detection of platelet derived growth factor (PDGF) in 50% blood serum.

FIG. 18 shows an AC voltammogram obtained from an electrochemical peptide-based (E-PB) sensor fabricated using click chemistry for HIV antibodies detection.

Like reference symbols in the various drawings indicate like elements.

### DETAILED DESCRIPTION

While many different versions of E-DNA sensors have been reported, the fabrication method of choice remains the conventional “two-step approach,” which involves direct adsorption of thiolated nucleic acid probes onto the gold surface. Although this sensor fabrication method produces stable self-assembled monolayer (SAM) and fully functional sensors, it lacks general surface modification versatility. Another drawback of the conventional “two-step approach” is that there is no guarantee that the specific biosensing element will form a well ordered SAM simply because it is tethered to a thiol moiety. A more convenient approach is to first assemble a SAM containing terminal reactive groups that may serve as a well-characterized platform onto which one can subsequently couple different biosensing probes to the monolayer. For these reasons, click chemistry is well-suited for use in the fabrication of E-DNA sensors.

“Click” chemistry was first introduced by Sharpless as a strategy to synthesize large oligomers from relatively simple building blocks with remarkable modularity and diversity (Kolb et al., 2001, Angew. Chem., 40:2004-21). The click reaction demonstrates unprecedented chemoselectivity, in which, in one embodiment, an alkyne moiety selectively reacts with an azide moiety under mild solution conditions. See, for example, U.S. Pat. No. 7,375,234, which describes click chemistry and is incorporated herein by reference. As described herein, the click chemistry approach has been adapted to the fabrication of E-DNA sensors and the performance of those sensors was compared to sensors fabricated via the conventional “two-step approach”. The click chemistry approach offers a significant advantage of versatility in sensor array fabrication and, in addition to E-DNA sensors, has been adapted to other folding-based electrochemical sensing platforms, including electrochemical aptamer-based (E-AB) and electrochemical peptide-based (E-PB) sensors.

Thus, as described herein, methods of making electrochemical biosensors using click chemistry is provided. In addition to E-DNA sensors, click chemistry also has been used to produce E-AB and E-PB sensors.

Initially, an electrochemical sensor is provided. Electrochemical sensors are used routinely to detect a target analyte in a sample. See, for example, U.S. Pat. Nos. 5,951,836; 6,432,723 and US 2007/0020641. Typically, an electrochemical sensor includes a working electrode (WE), a counter electrode (CE) and a reference electrode (RE), although a 2-component electrochemical sensor, in which the reference electrode additionally acts as the counter electrode, also can be used. One of the ways in which electrodes can be applied to a substrate is by screen-printing. See, for example, U.S. Pat. Nos. 4,185,131; 5,682,884; 5,727,548; and 5,820,551. The substrate onto which one or more electrodes are applied can be, for example, paper, plastic, ceramic, or glass. When screen-printing, the electrodes are typically produced using conductive inks Conductive inks can include, for example, silver, gold, carbon, dielectric polymer, or nickel. It is understood by those in the art that different conductive inks have varying stabilities in the presence of, for example, different ions. Accordingly, the particular conductive ink(s) used on an electrode can be selected based on the particular application (e.g., the sample to which the electrochemical sensor will be exposed). In one embodiment, the WE and the CE are screen-printed with conductive carbon inks and the RE is screen-printed with conductive silver chloride inks. In another embodiment, the electrodes are gold-plated as described in U.S. application Ser. No. 12/967,547, filed on Dec. 14, 2010 and incorporated herein by reference.

Next, a binding ligand is attached to the electrochemical sensor using click chemistry. As discussed herein, click chemistry refers to a multi-step synthesis, and was originally described by Sharpless' laboratory (Kolb et al., supra). Although the methods described herein are not limited to any particular type of click chemistry, the most common type of click chemistry is the azide alkyne Huisgen 1,3-dipolar cycloaddition using a Cu catalyst. In one embodiment of click chemistry, the surface of the electrochemical sensor is modified with an azide moiety, and one end of the binding ligand is modified with alkyne. In one embodiment of click chemistry, the surface of the electrochemical sensor is modified with an alkyne, and one end of the binding ligand is modified with an azide moiety. The alkyne can be, for example, a C₁₋₁₀ alkyne such as a C₃-alkyne. In either case, the binding ligand is “clicked” onto the surface of the electrochemical sensor in the presence of the Cu catalyst at room temperature.

In some embodiments, the electrochemical sensor includes a SAM prepared using a compound having a terminal azide moiety, such as 1-azidoundecan-11-thiol and a non-modified species, 11-mercapto-1-undecanol. The compound having the azide moiety is prepared in the SAM such that the moiety is available to undergo click chemistry with the binding ligand. As one of skill in the art would understand, the SAM also can be prepared using a compound having a terminal alkyne moiety. Such species can be purchased commercially or can be prepared using known synthetic methods.

As used herein, a binding ligand is a compound that is used as a probe for the presence of the target analyte and that undergoes a conformational change upon binding by the target analyte. As will be appreciated by those in the art, the particular binding ligand used will depend on the target analyte being detected. Binding ligands for a wide variety of analytes are known or can be readily identified using known techniques. For example, when the target analyte is a protein, the binding ligand can be an antibody or a fragment thereof (e.g., FAbs) or a nucleic acid that undergoes a conformational change upon binding by the target analyte; when the analyte is a metal ion, the binding ligand can be a traditional metal ion ligand or a chelator that undergoes a conformational change as a result of binding by the metal ion. Aptamers also are useful as binding ligands. Additional binding ligands suitable for use with the electrochemical sensors described herein can be found, for example, in U.S. Pat. No. 6,432,723 and US 2007/0020641. Virtually any compound can be used as a binding ligand provided that it undergoes a conformational or positional change upon binding by the target analyte. Binding ligands containing either an azide or alkyne moiety, as described herein, can be purchased commercially or may be prepared using known synthetic methods.

Nucleic acids are well known in the art and include DNA molecules and RNA molecules as well as DNA or RNA molecules containing one or more nucleotide analogs. Nucleic acids used in electrochemical sensors can be single-stranded or double-stranded, which is generally dictated by its intended use. Nucleic acids used in electrochemical sensors as described herein typically are at least 10 nucleotides in length (e.g., at least 20, 25, 30, 40, 50, 75, 80, or 95 nucleotides in length) and can be as many as one or several hundred bases in length (e.g., 100 bp, 350 bp, 500 bp, 800 bp, or 950 bp) or several thousand bases in length (e.g., 1000 bp, 2 kb, 3.5 kb, 4.2 kb, 5.0 kb, or more). In certain embodiments, nucleic acids used in electrochemical sensors can be about 15 bp-about 5 kb, about 15 bp-about 2.5 kb, about 15 bp-about 1000 bp, about 20 bp-about 1000 bp, about 20 bp-about 500 bp, about 25 bp-about 5 kp, about 25 bp-about 1 kb, or about 50 bp-about 1 kb in length. Methods of making or obtaining nucleic acids are routine to those skilled in the art. Representative methods of making or obtaining (e.g., isolating) nucleic acids include, for example, chemical synthesis, cloning, or PCR amplification.

Polypeptides are well known in the art and refer to multiple amino acids or amino acid analogues joined by peptide bonds. Polypeptides used in electrochemical sensors as described herein can be at least about 10 amino acids (aa) in length (e.g., at least about 12 aa, 15 aa, 20 aa, 25 aa, 30 aa, 40 aa, 50 aa, 60 aa, 70 aa, 80 aa, 90 aa, or 95 aa in length), at least about 100 aa in length (e.g., at least about 125 aa, 200 aa, 250 aa, 375 aa, 500 aa, 650 aa, 725 aa, 875 aa, or 900 aa in length), or at least about 1000 aa in length (e.g., at least about 1250 aa, 1500 aa, 1750 aa, 2000 aa, 2500 aa, 3000 aa, or more in length). For example, polypeptides can be about 10 aa-about 500 aa, about 20 aa-about 100 aa, or about 25 aa-about 50 aa in length. Polypeptides also can be a full-length protein expressed from a nucleic acid sequence (e.g., a gene). Polypeptides can be obtained (e.g., purified) from natural sources (e.g., a biological sample) by known methods such as DEAE ion exchange, gel filtration, and hydroxyapatite chromatography. Polypeptides also can be obtained by expressing a nucleic acid (e.g., from an expression vector). In addition, polypeptides can be obtained by chemical synthesis. The amount and/or purity of a polypeptide can be measured using any appropriate method, e.g., column chromatography, polyacrylamide gel electrophoresis, or HPLC analysis.

Aptamers also are well known in the art and can include both nucleic acids (e.g., oligonucleotides) and polypeptides. Aptamers refer to nucleic acids or polypeptides that bind to a specific target molecule, and typically are obtained by selection from a large random-sequence pool. However, natural aptamers also exist. Nucleic acid aptamers or polypeptide aptamers can be used in electrochemical sensors as described herein.

Depending on the particular binding ligand(s) used, the ionic strength of the buffer in which the click chemistry reaction takes place may need to be modified in order for the reaction to proceed efficiently. As those of skill in the art would understand, the ionic strength of a buffer can be manipulated by controlling the amount of one or more salts (e.g., NaCl, MgCl₂) in the buffer. For example, in certain instances, the amount of one or more salts in the buffer used in the click chemistry reaction may be increased to neutralize the negative charges of the backbone of a nucleic acid binding ligand. In other instances, the amount of one or more of the salts in the buffer may be adjusted to avoid, for example, secondary structure formation in the binding ligand. In addition, those of skill in the art would understand that polypeptides and proteins do not have the negatively charged backbone that nucleic acids have and, thus, are less tolerant to salt; similarly, because of the varying features of the different amino acids that make up polypeptides and proteins, the amount or type of one or more monovalent or divalent cations can be modified as necessary to optimize the attachment of the binding ligand to the electrochemical sensor.

To detect whether or not a binding ligand undergoes a conformational change, the binding ligand generally includes a redox tag. Redox tags are well known in the art and include, without limitation, purely organic redox labels, such as viologen, anthraquinone, ethidium bromide, daunomycin, methylene blue, and their derivatives, organo-metallic redox labels, such as ferrocene, ruthenium, bis-pyridine, tris-pyridine, osmium tris-bipyridine, cobalt tris-bipyridine, bis-imidizole, and their derivatives, and labels such as oxazine and derivatives thereof (e.g., ifosfamide and tetrahydro-1,4-oxazine).

The electrochemical biosensors described herein, produced using click chemistry, can be used to determine whether or not a target analyte is present in a sample. Samples can be biological samples (e.g., whole blood, blood serum, plasma, saliva, urine, cell lysates, tissue digests, or cell media), environmental samples (e.g., sea water, ground water, or soil samples), and food samples (e.g., milk, tissue samples, meat extracts, beer and other beverages). The electrochemical biosensors described herein can be contacted with any such sample and the redox potential measured to determine whether or not the target analyte is present.

One example of an electrochemical biosensor includes a nucleic acid binding ligand (E-DNA). Such a binding ligand can be used, for example, for the sequence-specific detection of a polymorphism. Among the recently developed DNA sensing platforms, the E-DNA sensor, which is the electrochemical equivalent of optical molecular beacons, exhibits notable sensitivity, specificity and operational convenience whilst also being fully electronic, readily reusable and able to work in complex, contaminant-rich samples.

In one embodiment, the E-DNA sensor includes a redox-tagged stem-loop DNA probe covalently attached to the gold pixel on the electrode. A signal can occur due to the binding-induced change in the conformation of the stem-loop probe and the efficiency with which the attached redox tag transfers electrons to the electrode. In the absence of any target analyte, the stem-loop structure can hold the redox tag in proximity to the electrode, enabling efficient electron transfer between the redox tag and the electrode and resulting in the generation of a measurable redox current. Upon binding by the target analyte (e.g., complementary target DNA), the double-stranded conformation of the probe DNA disrupts the stem-loop structure and forces the redox tag away from the electrode, impeding the electron transfer between the redox tag and the electrode and resulting in a detectable reduction in the measurable redox current. This type of E-DNA sensor is referred to as a signal-off sensor. In some implementations, a signal-on E-DNA sensor can be designed such that binding by the target analyte produces a conformational change that enhances the efficiency of the electron transfer between the redox tag of the stem-loop DNA probe and the electrode, leading to an increase in the measurable redox current.

Another example of an electrochemical biosensor includes a polypeptide or protein binding ligand (E-PB). An E-PB can include a redox-tagged polypeptide or protein covalently attached to an electrode. In some embodiments, a polypeptide may be bound to an electrode to be used as an electrochemical sensor so as to detect a target protein, e.g., an antibody. In other embodiments, an antibody or portion thereof may be bound to an electrode to be used as an electrochemical sensor to detect a target polypeptide or protein. In the absence of the target analyte, the conformation of the polypeptide or protein is such that the redox tag is positioned away from the electrode, impeding or disabling the electron transfer between the redox tag and the electrode and resulting in the generation of little or no measurable redox current. In the presence of the target analyte, the conformation of the polypeptide or protein forces the redox tag towards the electrode, enabling efficient electron transfer between the redox tag and the electrode and resulting in the generation of a measurable redox current. An electrical signal can occur due to the binding-induced change in the conformation of the binding ligand and the efficiency with which the attached redox tag transfers electrons to the electrode. As indicated above, this type of sensor can be referred to as signal-on sensor but, alternately, can be configured to be a signal-off sensor.

Another example of an electrochemical biosensor includes an aptamer binding ligand (E-AB). An E-AB can include a redox tagged aptamer covalently attached to an electrode. Since aptamers can be either nucleic acid- or polypeptide-based, the above-described changes in conformation to increase or decrease the transfer of electrons by the redox tag apply similarly to aptamers.

In some implementations, the electrochemical biosensors described herein can be configured as voltammetric electrochemical biosensors that utilize alternating current voltammetry in a solution or buffer that includes the sample. The use of voltammetry allows control of the potential (voltage) of an electrode in contact with an analyte while the resulting current is measured. For example, a voltammetric scan can obtain information about a target analyte from an electrochemical biosensor by measuring current at a first electrode (e.g., a working electrode), where the current results from the transfer of electrons between the electrode and the analyte. A second electrode (e.g., a reference electrode) generally is a half cell with a known reduction potential (voltage), which does not pass any current between it and the analyte and acts as a reference in measuring and controlling the potential at the first electrode. A third electrode (e.g., a counter electrode) can pass the current needed to balance the current observed at the first electrode (e.g., the counter electrode).

Alternating current voltammetry (ACV), for example, can include superimposing a small alternating voltage of a constant magnitude on a voltammetric scan. A plot of alternating current verses the voltage applied to a working electrode provides a qualitative measure of the electrode process. In some implementations, low frequency (e.g., 10 Hz.) small alternating voltages (e.g., 25 millivolts (mV)) can provide maximum peak resolution. Other forms of voltammetry can also be used that can include, but are not limited to, cyclic voltammetry, linear sweep voltammetry, normal pulse voltammetry, differential pulse voltammetry, and square wave voltammetry.

The electrochemical biosensors described herein can include one or more binding ligands that recognize a single target analyte or, in alternative embodiments, an electrochemical sensor as described herein can include two or more different binding ligands that recognize at least two different target analytes. Microarray technology is well known in the art, and can be similarly applied to the placement of binding ligands on an electrochemical biosensor as described herein.

The electrochemical sensors produced using click chemistry as described herein have been shown to be reusable; that is, a binding ligand attached to an electrochemical sensor using click chemistry can be stably and repeatedly regenerated by washings. However, an electrochemical biosensor as described herein can be configured to be disposable (e.g., single-use).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Folding-Based Electrochemical DNA Sensor Fabricated by “Click” Chemistry

Part A—Materials and Methods

Materials and Instrumentation. The reagents, 11-mercapto-1-undecanol (C11-OH), 8 M guanidine hydrochloride (GHCl), tris-(2-carboxyethyl) phosphine hydrochloride (TCEP), trizma base, copper sulfate (CuSO₄), sodium ascorbate, ethanol (EtOH), dimethyl sulfoxide (DMSO) and tris[(1-benzyl-1H-1,2,3-triazol-4-yl)methyl]amine (TBTA), were used as received (Sigma-Adrich, St. Louis, Mo.). Azide-terminated undecyl disulfide was obtained from Asemblon, Inc. (Redmond, Wash.) and was used without further purification. Human whole blood purchased from Innovative Research (Novi, Mich.) was used as received. All other chemicals were of analytical grade. All the solutions were made with deionized water (DI water) purified through a Milli-Q system (18.2 MΩ·cm, Millipore, Bedford, Mass.). Physiological buffer solution (Phys2, pH 7.4) consisted of 20 mM Tris, 140 mM NaCl, 5 mM KCl, 1 mM MgCl₂, 1 mM CaCl₂ adjusted to pH 7.4 with hydrochloric acid.

For both click chemistry-based and conventional E-DNA sensors, a methylene blue (MB)-modified stem-loop oligonucleotide complementary to the K-ras gene was used as the probe with the sequence 5′-CCG TTA CGC CAC CAG CTC CAA ACG G-(CH₂)₇—NH-MB-3′ (SEQ ID NO:1) (Biosearch Technologies, Inc. Novato, Calif.). The MB redox moiety was conjugated to the 3′ end of the oligonucleotide via succinimide ester coupling to a 3′-amino modification. The click chemistry probe DNA was modified with a C3-alkyne at the 5′-end for conjugation to the surface azide and the probe sequence was as follows: 5′ HCC≡C—(CH₂)₃-CCG TTA CGC CAC CAG CTC CAA ACG G-(CH₂)₇—NH-MB-3′ (SEQ ID NO:2).

The conventional E-DNA sensor required a probe with a direct thiol modification at the 5′-end and the sequence was as follows: 5′ HS—(CH₂)₁₁—CCG TTA CGC CAC CAG CTC CAA ACG G-(CH₂)₇—NH-MB-3′ (SEQ ID NO:3).

The K-ras is a gene that encodes one of the proteins in the epidermal growth factor receptor (EGFR) signaling pathway. Pancreatic and lung cancers harbor high incidences of K-ras mutant alleles, and these mutations are early events in colorectal tumor development. The detection of K-ras mutations enables understanding of cancer biology and pathogenesis. The target DNA sequence (WT-Gly) was obtained via commercial synthesis (polyacrylamide gel electrophoresis purification, Integrated DNA Technologies, Coralville, Iowa), and the sequence was as follows: WT-Gly: 5′-TTG GAG CTG GTG GCG TA-3′ (SEQ ID NO: 4).

Electrochemical measurements were performed at room temperature (22±1° C.) using a CHI 1040A Electrochemical Workstation (CH Instruments, Austin, Tex.). Polycrystalline gold disk electrodes (CH Instruments, Austin, Tex.) with geometric area of 0.0314 cm² were used as working electrodes. The counter electrode used was a platinum wire electrode and an Ag/AgCl (3M KCl) electrode served as the reference electrode, both from CH Instruments (Austin, Tex.). Prior to sensor fabrication, the gold electrodes were polished with a 0.1 μm diamond slurry (Buehler, Lake Bluff, Ill.), rinsed with DI water and sonicated in a low power sonicator for approximately five minutes to remove bound particulates. They were then electrochemically cleaned by a series of oxidation and reduction cycles in 0.5 M H₂SO₄ and in 0.05 M H₂SO₄. The real area of each electrode was determined from the charge associated with the gold oxide stripping peak obtained after the cleaning process.

Preparation of a “Click” Chemistry-based E-DNA Sensor. Prior to sensor fabrication, azide-terminated undecyl disulfide was first reduced to 1-azidoundecan-11-thiol (C11-N₃) in the presence of TCEP. A cleaned gold electrode was placed in a 200 μM solution of C11-N₃ prepared in 3:1 EtOH/H₂O for 10 min. The electrode was subsequently incubated in a 2 mM C11-OH solution in 4:1 EtOH/H₂O for ˜2.5 hrs to complete the monolayer formation. The mixed monolayer-modified electrode was then rinsed with copious amount of DI water, dried with N₂ gas, and transferred to a click mixture containing 900 μM TBTA, 400 μM sodium ascorbate, 400 μM CuSO₄ and 3.5-4 μM of alkyne-modified probe DNA in 1:1 DMSO/H₂O. The click reaction was allowed to proceed for 30 min in dark. To remove physically adsorbed probe DNA, the electrode was rinsed thoroughly with DI water, 5% tween, EtOH and then again in the reverse order. Prior to transferring the sensor electrode to the electrochemical cell for target interrogation, the electrode was rinsed with DI water for 30 sec.

Two additional sensors were fabricated to evaluate the extent of non-specific adsorption of probe DNA during the click reaction. The two sensors were fabricated following the above procedures, with the exception that one sensor was fabricated in the absence of CuSO₄, whereas the other sensor was constructed without sodium ascorbate, the reducing agent.

Preparation of a Conventional E-DNA Sensor. Prior to Sensor Fabrication, the Probe DNA was first reduced to its free thiol form in presence of TCEP. A cleaned gold electrode was first immersed in a 2 mM C11-OH ethanolic solution for 5 min. The electrode was subsequently immersed in a Phys2 buffer containing 2 μM probe DNA for 2 hr. The sensor electrode was treated with a 30-sec DI water rinse prior to introduction to the electrochemical cell for target interrogation.

Electrochemical Measurements. E-DNA sensor performance was analyzed by alternating current voltammetry (ACV). Alternating current (AC) voltammograms were recorded in a Phys2 buffer or 100% human whole blood from 0 V to −0.5 V vs. Ag/AgCl with a 10 Hz, 25 mV AC potential. Prior to target interrogation, the electrodes were allowed to equilibrate in a Phys2 buffer for at least 20 minutes. The E-DNA sensor response to the target DNA was measured by incubating the electrodes in 1 μM of the WT-Gly target DNA (in a Phys2 buffer or human whole blood). The sensors were interrogated at different intervals in the target solution until a stable peak current was obtained. The ratio between the stabilized peak current in the target DNA solution and the peak current in the target DNA-free solution was used to calculate the signal suppression caused by the target.

Sensor regeneration was achieved by rinsing for 30 sec with deionized water for sensors utilized in Phys2 buffer or by incubating with 4 M GHCl for 4 min, followed by rinsing with deionized water for 30 sec for sensors utilized in whole blood. Sensor regeneration was verified by AC voltammogram recorded 5 minutes after re-immersion in the buffer or whole blood.

The number of electroactive DNA probes on the electrode surface, Nₜₒₜ was determined using a previously established relationship with ACV peak current described in eq. 1:

I_{avg}(E₀)=2nfFNₜₒₜ sin h(nFE_{ac}/RT)/[cos h(nFE_{ac}/RT)+1] (Eq. 1)

Where I_{avg}(E₀) is the average AC peak current in a voltammogram, n is the number of electrons transferred per redox event (n=2, MB label), F is the Faraday current, R is the universal gas constant, T is the temperature, E_{ac} is the peak amplitude, and f is the frequency of the applied ACV. The surface density of DNA probes was measured in the number of electroactive DNA probes per unit area.

Part B—Results

This Example describes a folding-based electrochemical DNA (E-DNA) sensor fabricated by Sharpless click chemistry. The click chemistry-based E-DNA sensor compares favorably to a sensor fabricated via the conventional “two-step approach,” which involves direct adsorption of thiolated probe DNA onto the gold electrode surface. The click chemistry-based sensor is equally selective and can be employed directly in undiluted human whole blood.

The E-DNA sensor is comprised of a redox-labeled DNA stem-loop probe covalently attached to a gold disk electrode. In the absence of target, the stem-loop conformation holds the redox label in close proximity to the electrode, facilitating electron transfer. Upon binding to a complementary DNA target, hybridization forces the redox tag away from the electrode, impeding electron transfer and producing a reduction in redox current that is readily observable.

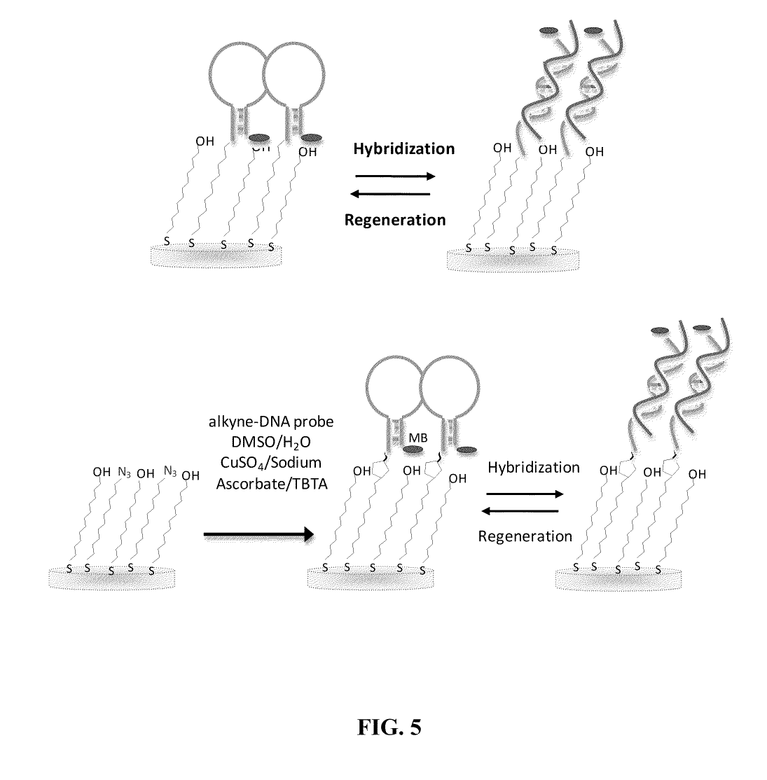

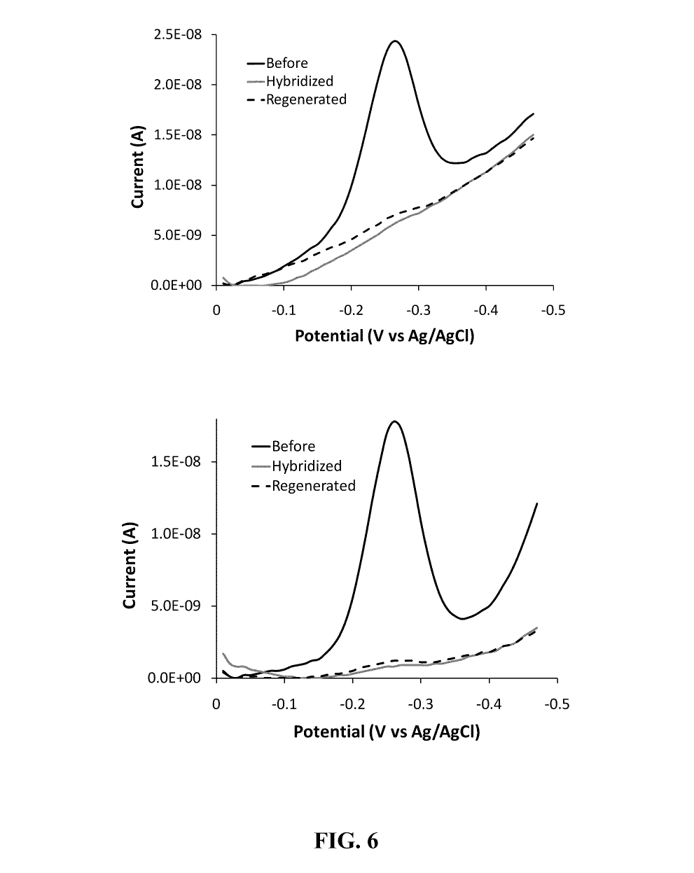

A 25-base stem-loop DNA probe with the 15-base loop region targeting the K-ras gene was utilized. The two 5-base sequences at the termini formed the double-stranded stem of the DNA probe (Lai et al., 2007, Anal. Chem., 79:229-33; Gerasimov et al., 2010, Chem. Commun., 46:395-7). For the click chemistry-based E-DNA sensor, the DNA probe was modified at the 5′-end with a C3-alkyne, whereas the 3′-end is modified with the redox label, methylene blue (MB). The first step in fabricating the sensor involved the formation of a mixed SAM consisted of 1-azidoundecan-1′-thiol and 11-mercapto-1-undecanol on a gold disk electrode. The alkyne-modified DNA probes were subsequently conjugated to the azide-containing SAM in presence of CuSO₄, sodium ascorbate and tris-(benzyltriazolylmethyl)amine (FIG. 5). For the conventional E-DNA sensor, the probe design was essentially identical to the probe used in the click chemistry-based sensor, with the exception that the probe was modified with an 11-carbon thiol at the 5′-end for direct attachment to the gold electrode (FIG. 5). Fabrication of a conventional E-DNA sensor involved the formation of a 11-mercapto-1-undecanol SAM, followed by the incorporation of 11-carbon thiolated DNA probes into the SAM. While the two fabrication approaches are relatively different, sensor preparation time for both sensors was ˜3 hrs. The click chemistry-based sensor displayed an average surface probe density of 8.3×10¹¹ molecules cm⁻² while the conventional sensor showed an average density of 7.6×10¹¹ molecules cm⁻².

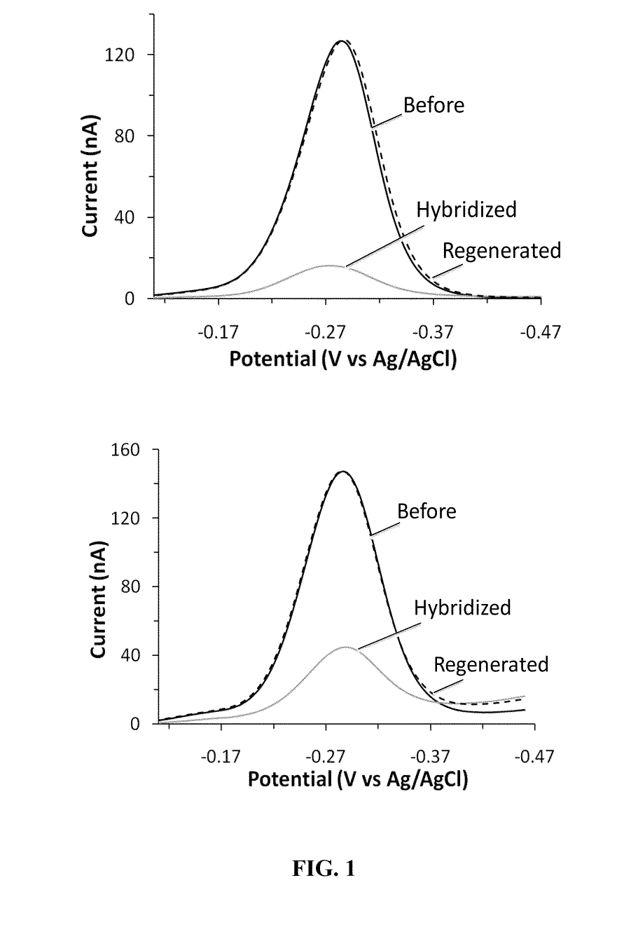

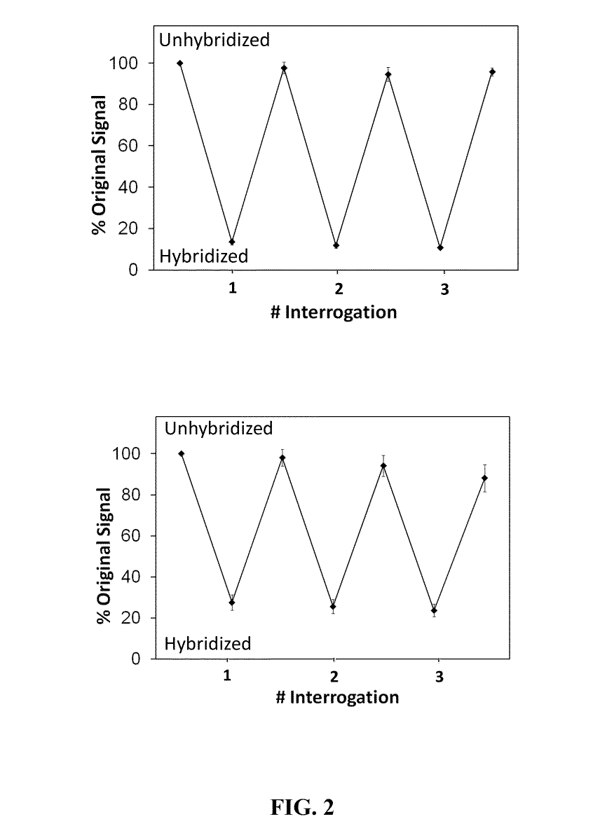

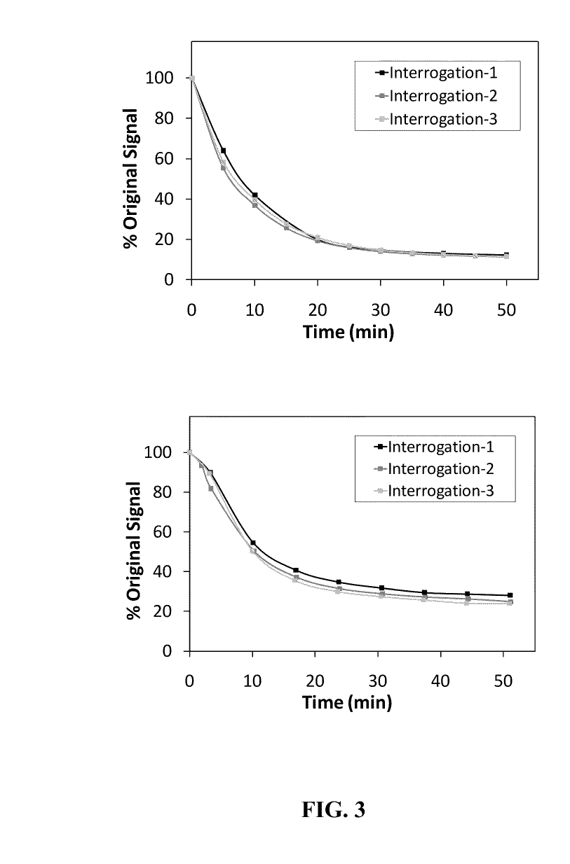

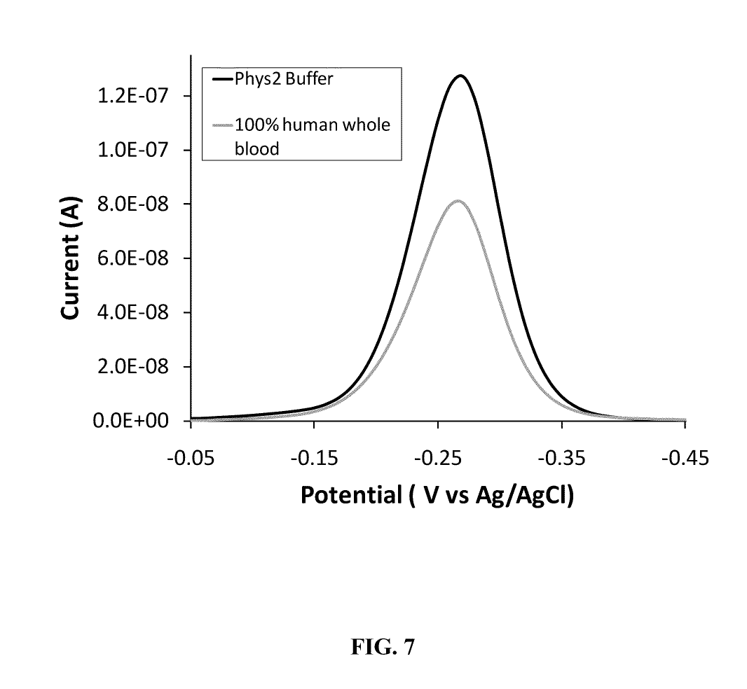

Sensor performance was assessed by alternating current voltammetry (ACV) by scanning from 0 to −0.5 V (vs. Ag/AgCl/3M KCl) in a physiological buffer (Phys2, pH 7.4). For the conventional E-DNA sensor, in absence of the target DNA, a sharp, well-defined AC voltammetric peak consistent with the ˜−0.28 V reduction potential of the methylene blue (MB) redox moiety was observed (FIG. 1, top). For the click chemistry-based sensor, a slightly more negative reduction potential (˜−0.29 V) was observed, owing to minor differences in the reference electrode employed (FIG. 1, bottom). In the presence of 1.0 μM full-complement target DNA, a robust 87% decrease in the MB reduction current we observe for the conventional E-DNA sensor and a similar response (74%) was observed for the click chemistry-based sensor. While the probe density between the two sensors did not differ significantly, the lower signal suppression observed in the click chemistry-based sensor was probably due to the lack of probe conformation homogeneity. Upon sensor regeneration via a 30 sec room-temperature deionized water rinse, both sensors exhibit less than 0.5% of initial current loss, supporting the overall similarity between the two sensors. Since sensor regeneration is a characteristic of the E-DNA sensor, this indicates that the observed MB current arises from probes specifically conjugated onto the SAM surface and not originated from physically adsorbed DNA probes. To further support this conclusion, two separate control experiments were conducted. Two sensors were fabricated; one in the absence of CuSO₄ and one in the absence of sodium ascorbate, the reducing agent. Omission of either of the active components results in the lack of Cu(I) catalyst and, hence, prevents the click reaction from proceeding. As a result, both sensors showed negligible MB reduction current when compared to sensors fabricated without the omissions (FIG. 6). While non-specific adsorption of DNA probes cannot completely be eliminated, continuous scanning at the set potential range led to the removal of most of the non-specifically adsorbed probes. The ability to reuse a single sensor multiple times is a unique and valuable attribute of the E-DNA sensor. Here, the two sensors were compared in sensor performance reproducibility per interrogation and sensor fabrication reproducibility. The conventional E-DNA sensor exhibited a mean signal regeneration of 98.4±1.5% per use (FIG. 2, top). The sensor signal also was highly reproducible, producing a mean signal suppression of 88.3%±0.5% when challenged with 1.0 μM full-complement target. The E-DNA sensor fabricated via click chemistry was comparably reusable and reproducible; 99.7±4.5% signal recovery per wash was achieved with a 73.0±1.4% signal drop per use over three iterations (FIG. 2, bottom). Notably, each plot displayed the average data obtained from three different sensors fabricated on three different electrodes, suggesting general reproducibility in the sensor fabrication methods. This, however, showed that the click chemistry-based sensor was only slightly less reproducible with regards to sensor fabrication reproducibility.

For the E-DNA sensor, response time is another indicator of sensor performance. As shown in FIG. 3, under the same experimental condition (i.e., target DNA concentration, hybridization temperature etc), both sensors reached signal saturation in approximately 30 min. More importantly, for the conventional sensor, identical hybridization kinetics were observed among the three interrogations, and only very minor differences were noticeable in the first 10 min of the hybridization. The click chemistry-based sensor, however, showed slightly less reproducibility among interrogations. The hybridization kinetics observed in the first interrogation is slightly sluggish when compared to the second or third interrogation, owing to the lack of homogeneity in the probe conformation when used immediately after the click reaction. Post-reaction annealing will, presumably, circumvent this problem. In short, the overall response time of both sensors was very similar, suggesting that the resulting 1,2,3-triazole moiety in the click chemistry-based sensor poses negligible adverse effects on hybridization kinetics.

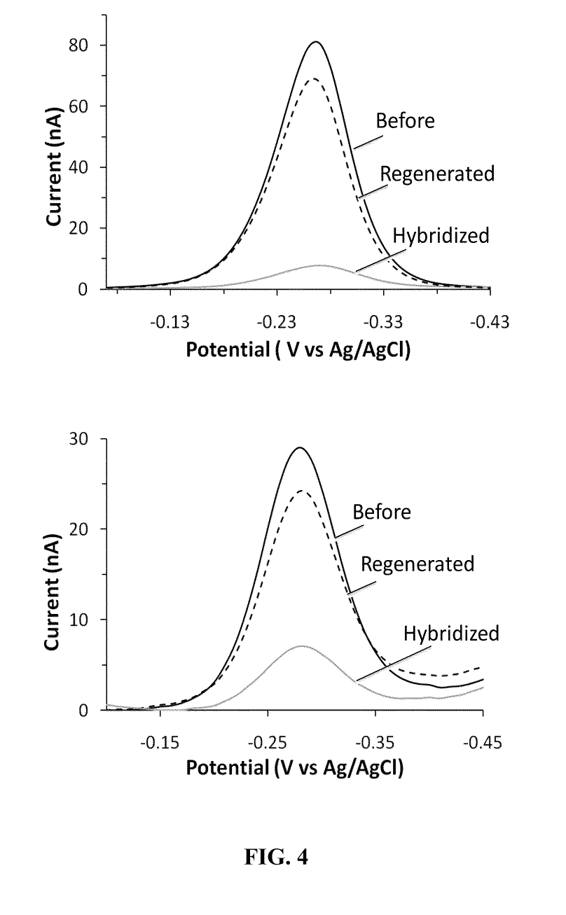

Of greater relevance to real-world sensing scenarios is that both sensors fared well, even when challenged with contaminant-ridden samples. FIG. 4 shows both sensors' response in 100% human whole blood. A ˜37% initial signal reduction was observed when the sensor was first introduced into the whole blood matrix (when compared to current observed in a Phys2 buffer) (FIG. 7). The reduction in current was probably due to the differences in the viscosity and ionic strength, which is known to affect the MB current in E-DNA sensors. No peak potential shift in the MB redox potential was observed. More importantly, in the presence of 1.0 μM full-complement target DNA, a 90% decrease in the MB reduction current was observed for the conventional E-DNA sensor and a similar response (77%) for the click chemistry-based sensor. Upon sensor regeneration via a simple 4-min room temperature incubation in 4 M guanidine-hydrochloride (GHCl), both sensors exhibited a loss of ˜15% of the initial current, presumably caused by activities of the nucleases present in the blood. Nonetheless, both sensors demonstrated stability in human whole blood, showing less than 15% initial current loss over a course of 4 hours. The sensors' response (i.e. signal suppression, binding kinetics) in whole blood was comparable to that observed in a clean buffer system, supporting potential real world application of this sensor. While the data presented here were obtained from male whole blood, female whole blood was also utilized and similar results were obtained.

It was concluded that the E-DNA sensors fabricated via click chemistry showed comparable attributes compared to sensors fabricated via the conventional “two-step approach”. Therefore, this versatile sensor fabrication approach can be implemented in the fabrication of cost-effective E-DNA sensor arrays for point-of-care medical diagnosis. Based on this study, the click chemistry approach could presumably be adapted to the fabrication of E-AB and E-PB sensors for the detection of proteins and small molecules.

### Example 2

### Fabrication of an Electrochemical DNA Sensor Array Via Potential-Assisted “Click” Chemistry

Part A—Materials and Methods

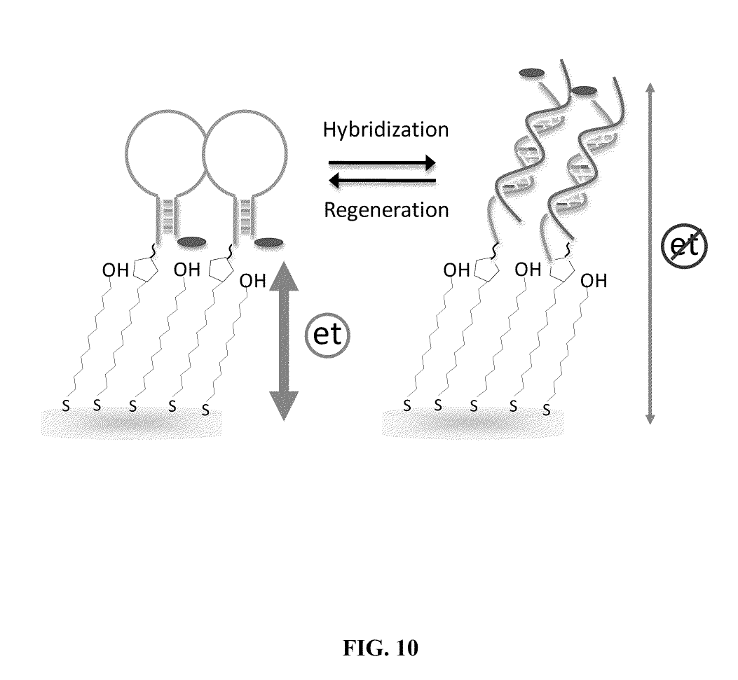

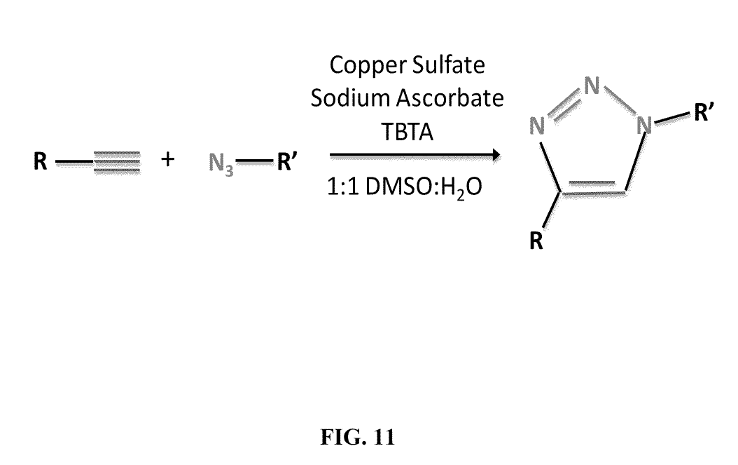

Materials and Instrumentation. The reagents, 11-mercapto-1-undecanol (C11-OH), sulfuric acid (H₂SO₄), trizma base, copper sulfate (CuSO₄), ethanol (EtOH), TWEEN® 20 (Tween), dimethyl sulfoxide (DMSO) and tris[(1-benzyl-1H-1,2,3-triazol-4-yl)methyl]amine (TBTA) were used as received (Sigma-Adrich, St. Louis, Mo.). 11-azido-1-undecanol (C11-N3) was obtained from ProChimia Surfaces (Sopot, Poland) and was used without further purification. The surrogate probe, alkyne-modified C7-MB, was obtained from Biosearch Technologies (Novato, Calif.) (FIG. 10). All other chemicals were of analytical grade. All the solutions were made with deionized water (DI water) purified through a Milli-Q system (18.2 MΩ·cm, Millipore, Bedford, Mass.). Physiological buffer solution (Phys2, pH 7.4) consisted of 20 mM Tris, 140 mM NaCl, 5 mM KCl, 1 mM MgCl₂, 1 mM CaCl₂ adjusted to pH 7.4 with hydrochloric acid.

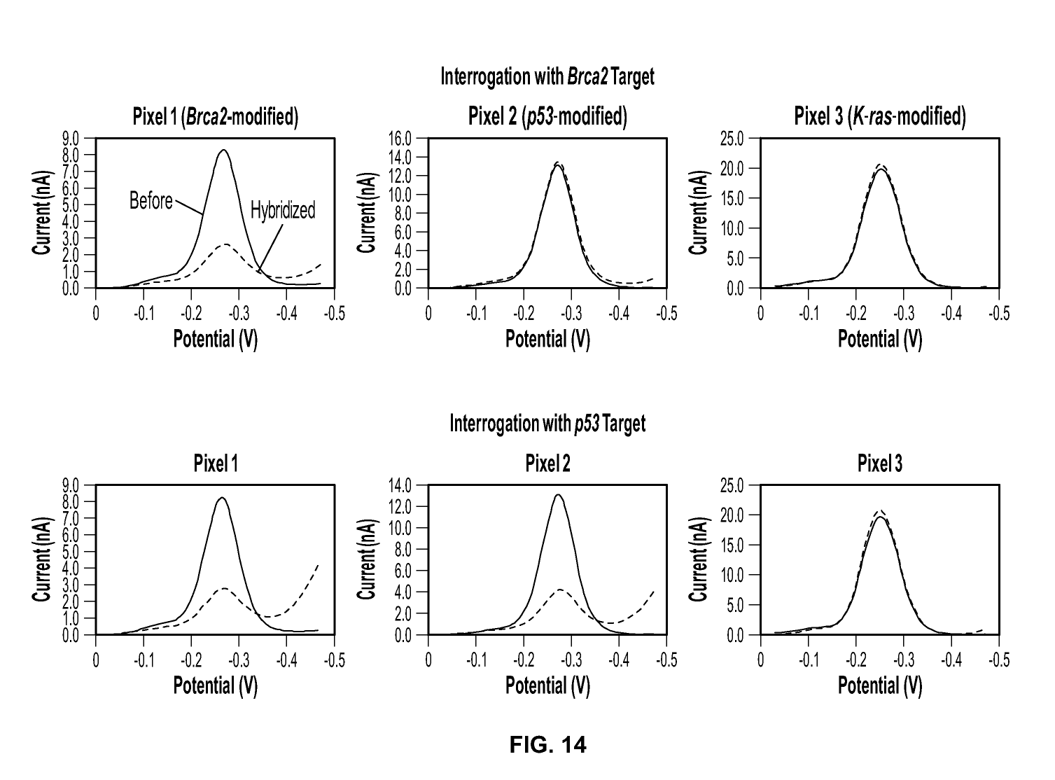

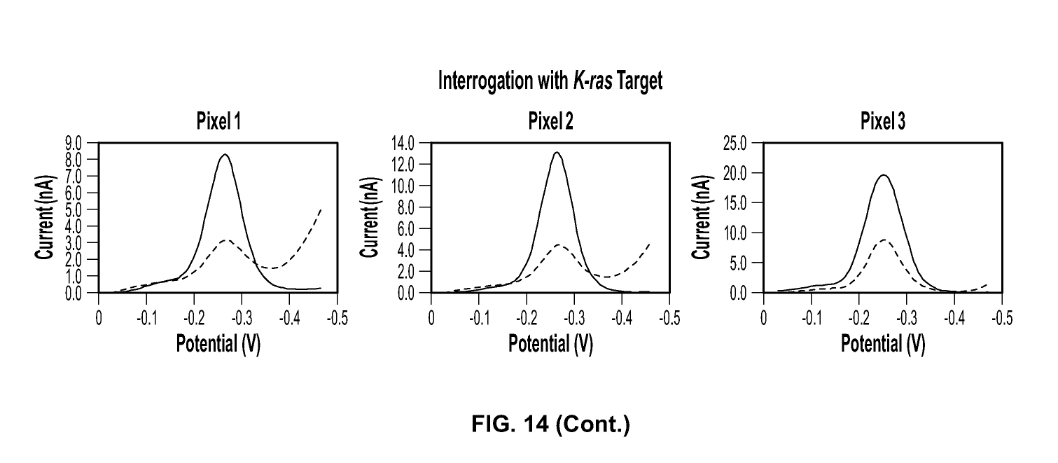

For the E-DNA sensor array, dual-modified oligonucleotides obtained from Biosearch Technologies, Inc. (Novato, Calif.) were used as the DNA probes (FIG. 14). A methylene blue (MB) redox reporter was conjugated to the 3′ end of the oligonucleotide via succinimide ester coupling to a 3′-amino modification. The probe was also modified with a C3-alkyne at the 5′-end for conjugation to the surface azide. The probe sequences used are as follows:

K-ras probe:
(SEQ ID NO: 5)
5′-alkyne-CCGTTACGCCACCAGCTCCAAACGG-Methylene
Blue- 3′
p53 probe:
(SEQ ID NO: 6)
5′-alkyne-CCGTTCCTCCGGTTCATGCCAACGG-Methylene
Blue-3′
Brca2 probe:
(SEQ ID NO: 7)
5′-alkyne-CCGTTACGGCCCTGAAGTACAACGG-Methylene
Blue-3′

The target DNA sequences were purchased from Integrated DNA Technologies (Coralville, Iowa). The target sequences used are as follows:

| | |
|---|---|
| K-ras target: | |
| 5′-TTGGAGCTGGTGGCGTA-3′ | (SEQ ID NO: 8) |
| p53 target: | |
| 5′-TGGCATGAACCGGAGGA-3′ | (SEQ ID NO: 9) |
| Brca2 target: | |
| 5′-TGTACTTCAGGGCCGTA-3′ | (SEQ ID NO: 10) |

Electrochemical measurements were performed at room temperature (22±1° C.) using a CHI 1040A Electrochemical Workstation (CH Instruments, Austin, Tex.). Cyclic voltammetry (CV) and alternating current voltammetry (ACV) were both employed in this study. Polycrystalline gold disk and glassy carbon electrodes (CH Instruments, Austin, Tex.) were used as working electrodes. The counter electrode used was a platinum wire electrode and a Ag/AgCl (3 M KCl) electrode served as the reference electrode, both from CH Instruments (Austin, Tex.). Prior to sensor fabrication, the gold electrodes were polished with a 0.1 μm diamond slurry (Buehler, Lake Bluff, Ill.), rinsed with DI water and sonicated in a low power sonicator for approximately five minutes to remove bound particulates. They were then electrochemically cleaned by a series of oxidation and reduction cycles in 0.5 M H₂SO₄ and in 0.05 M H₂SO₄. The real area of each electrode was determined from the charge associated with the gold oxide stripping peak obtained after the cleaning process.

Cyclic Voltammetric Studies of Cu²⁺ in Presence of TBTA in 1:1 DMSO/H₂O. Glassy carbon electrodes were polished using 0.1 μm diamond slurry (Buehler, Lake Bluff, Ill.), rinsed with DI water, sonicated for approximately five minutes, and were dried under nitrogen (N₂). Gold disk electrodes were cleaned accordingly to a similar procedure, with the addition of an electrochemical cleaning step. An azide-containing self-assembled monolayer (SAM) was developed on the electrode by first incubating the electrode in a solution consisted of 67 mM C11-OH and 133 mM C11-N3 for 30 minutes. The electrodes were subsequently immersed in 5 mM C11-OH for another 4 hrs to complete the SAM formation. Cyclic voltammograms were recorded at a scan rate of 100 mV/s in a deacerated DMSO/H₂O solution containing 75 mM CuSO₄, 150 mM TBTA and 0.1 M NaCl.

“Click” Potential Optimization. Three different applied potentials were chosen (i.e., −300, −400 and −500 mV) to optimize the number of captured probes. The azide-containing SAM-modified electrodes were first immersed in a DMSO/H₂O solution containing 75 mM CuSO₄, 150 mM TBTA, 0.1 M NaCl and 3.6 μM alkyne-modified C7-MB. The assigned potential was then applied to the electrodes for a fixed 30 minutes. To remove physically adsorbed probe DNA, the electrode was rinsed thoroughly with DI water, 5% Tween, EtOH and then again in the reverse order. Electrochemical characterization of the films was performed using ACV. AC voltammograms were recorded in a Phys2 buffer from −0.05 V to −0.5 V vs. Ag/AgCl with a 10 Hz, 25 mV AC potential.

“Click” Efficiency Differences Between C7-MB and the K-ras DNA Probe. The C7-MB and K-ras probes were conjugated on azide-modified electrodes via potential-assisted click chemistry at the optimized click potential of −500 mV. The click mixture used was a 1:1 DMSO/H₂O solution containing 75 mM CuSO₄, 150 mM TBTA, 0.1 M NaCl and 3.6 μM alkyne-modified C7-MB. Electrochemical characterization of the resultant films was performed using ACV. AC voltammograms were recorded in a Phys2 buffer from −0.05 V to −0.5 V vs. Ag/AgCl with a 10 Hz, 25 mV AC potential.

Surface Probe Density Calculations. The number of electroactive probes (e.g., C7-MB, K-ras DNA probes) on the electrode surface, Nₜₒₜ was determined using a previously established relationship with ACV peak current described in eq. 1:

I_{avg}(E₀)=2nfFNₜₒₜ sin h(nFE_{ac}/RT)/[cos h(nFE_{ac}/RT)+1] (Eq. 1)

Where I_{avg}(E0) is the average AC peak current in a voltammogram, n is the number of electrons transferred per redox event (n=2, MB label), F is the Faraday current, R is the universal gas constant, T is the temperature, E_{ac} is the peak amplitude, and f is the frequency of the applied ACV. The surface density of probes was measured as the number of electroactive probes per unit area.

Fabrication of a 3-pixel E-DNA Sensor Array. The 3-pixel gold electrode array used in this study was fabricated using standard microfabrication techniques as published elsewhere (Sassolas et al., 2008, Chem. Rev., 108:109). The electrode array was modified with an azide-containing SAM as described above. The electrode array was then immersed in a click mixture containing 1.4 mM TBTA, 0.44 mM CuSo₄, 4.3 μM Brca2 probe, 0.1 M NaCl, and 2.7 mM MgCl₂. Only Pixel 1 of the array was biased at −500 mV (vs. Ag/AgCl reference electrode) for 30 minutes; the remaining two pixels were not connected to the potentiostat (i.e. open circuit potential). The array was then removed from the solution, rinsed with DI water, 5% Tween, EtOH and then again in the reverse order. Next, the array was immersed in the second click mixture containing the p. 53 probe for 30 minutes, with Pixel 2 biased at −500 mV while keeping Pixel 1 and 3 disconnected from the potentiostat. After rinsing, the electrode array was incubated in the third click mixture containing the K-ras probe. Pixel 3 was held at −500 mV for 30 minutes, whereas Pixels 1 and 2 were disconnected.

Target Interrogation and Sensor Regeneration. The resultant 3-pixel E-DNA sensor array was allowed to equilibrate in a Phys2 buffer until a stable baseline was observed in the AC voltammogram. Sensor interrogation with 1 μM target DNA was then performed in a sequential manner according to the following order: Brca2, p. 53, K-ras. To determine the hybridization kinetics, each sensor in the array was interrogated at different time intervals in the target solution until a stable peak current was obtained (i.e. signal saturation). The ratio between the stabilized peak current in the target DNA solution and the peak current in the target DNA-free solution was used to calculate the signal suppression caused by the target. Post-hybridization, sensor regeneration was achieved by rinsing for 30 seconds with deionized water. The sensor array was placed back into a Phys2 buffer to determine the degree of sensor regeneration.

Part B—Results

The fabrication of a 3-pixel electrochemical DNA sensor array via potential-assisted click chemistry is discussed herein. It was found that the sensors in the array exhibit close to identical sensor performance when compared to sensors constructed via conventional click chemistry.

In a standard click chemistry reaction, sodium ascorbate is used to reduce Cu(II) to Cu(I), and the ligand, tris[(1-benzyl-1H-1,2,3-triazol-4-yl)methyl]amine (TBTA), is used to stabilize the Cu(I) catalyst (FIG. 11). A less explored but equally advantageous application of this technique is to use electrochemistry to activate the copper catalyst directly on a self-assembled monolayer (SAM)-modified electrode. This allows precise control of the site-specific modification of closely spaced electrode elements with target molecules. While other site-specific electrode modification techniques have been utilized in sensor-related applications, the potential-assisted click chemistry approach, however, has not been employed in the fabrication of biosensors to date. Of note, since the generation of Cu(I) is potential-dependent, potential-assisted click chemistry can also be utilized in the fabrication of a multi-pixel sensor array by varying the potential applied to each pixel. This approach can be implemented in the fabrication of biosensor arrays for simultaneous detection of multiple clinically-relevant targets for point-of-care diagnosis.

For the first time, the fabrication of a 3-pixel E-DNA sensor array is described with each pixel functionalized with a different stem-loop DNA probe utilizing potential-assisted click chemistry. The strategy was to first optimize the parameters of potential-assisted click chemistry on SAM-modified electrodes via a surrogate probe and, subsequently, use these parameters to fabricate the sensor array. DNA hybridization efficiency and kinetics were used to characterize the sensors, and results were compared to the E-DNA sensors constructed via conventional click chemistry (Immoos et al., 2004, J. Am. Chem. Soc., 126:10814). First, the electrochemical reduction of Cu(II) to Cu(I) in the presence of TBTA was investigated using a glassy carbon electrode (GC) and a SAM-modified gold disk electrode (in the solvent system later used in the fabrication of the E-DNA sensor array). The SAM used in this study was a monolayer of 11-mercapto-1-undecanol.

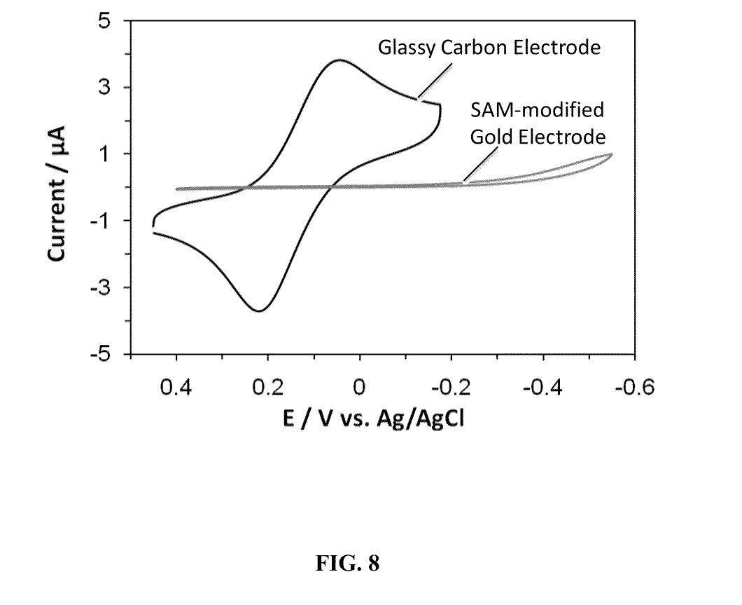

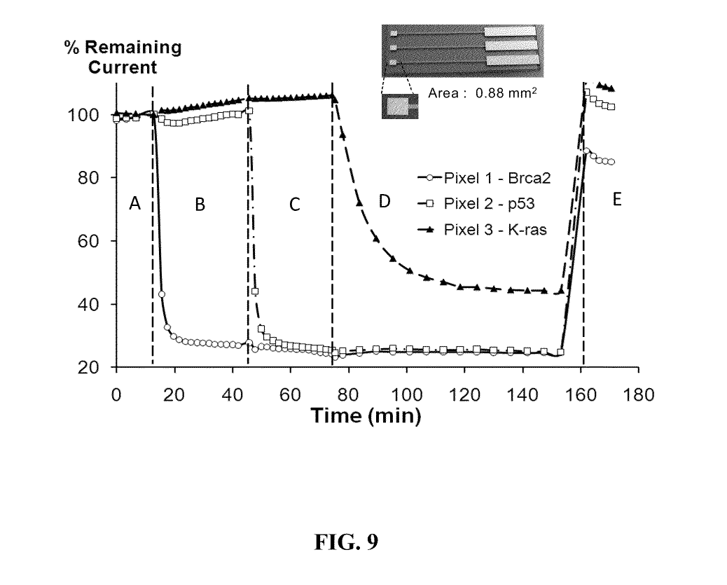

(C11-OH) and 1-azido-11-undecanethiol (C11-N3), similar in composition to the azide-containing SAM used previously (Immoos et al., supra). In order to determine the reduction potential of Cu(II) in presence of TBTA, cyclic voltammetry was performed in a deacerated solution of CuSO₄ and TBTA in 1:1 dimethyl sulfoxide (DMSO)/H₂O supplemented with 0.1 M NaCl. As observed, a reversible voltammetric wave with a half-wave potential of +50 mV was evident for the bare GC electrode within the scan range of 450 mV and −175 mV, indicating the reversible reduction of Cu(II) to Cu(I) in the presence of TBTA (FIG. 8). These results validate the previously reported electrochemical reversibility of the Cu(II)/Cu(I) reaction in DMSO. Therefore, it is apparent, rom the results presented herein, that adequate amount of Cu(I) catalyst can be electrochemically generated through a C11-0H/C11-N3 SAM-modified gold electrode.

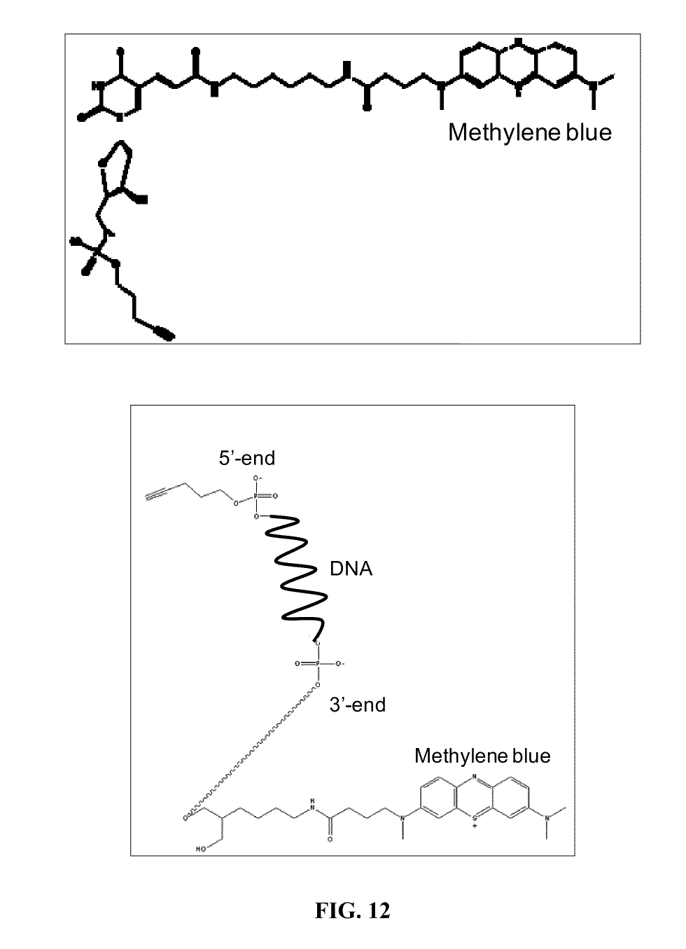

Having established the feasibility of potential-assisted generation of Cu(I) in a solvent system, the click potential-dependency on probe capture was investigated. A surrogate probe, an alkyne-modified methylene blue (MB) (C7-MB), was used (FIG. 12). Here, the applied potential was varied while keeping all other experimental conditions constant, with the goal of determining an optimal potential for probe immobilization. The probe density at each applied potential was then calculated utilizing a previously established method (Yang et al., 2009, Chem. Comm., 20:2902; Ricci et al., Chem. Commun., 36:3768). The computed probe densities at −300, −400 and −500 mV (mV vs. Ag/AgCl reference electrode) were 6.88×10¹¹, 1.02×10¹² and 1.88×10¹² molecules/cm², respectively. Despite the apparent trend that more negative applied potentials yielded higher probe densities, no attempt was made to utilize potentials more negative than −500 mV to prevent reductive desorption of the monolayer. For this system, the computed C7-MB probe density at −500 mV was almost 3 times higher than that at −300 mV, a previously reported applied potential for electrochemical-assisted click chemistry (Xiao et al., 20006, PNAS USA, 103:16677-80; Cash et al., 2009, Anal. Chem., 81:656; Kolb et al., 2001, Angew. Chem., 40:2004). Based on this study, −500 mV appeared to be a good compromise between probe coverage and SAM stability, and, thus, was used in the remaining studies. Of note, control experiments conducted at open circuit potential (OPC) yielded a notably lower probe density (9.28×10⁹ molecules/cm²), suggesting that a relatively negative potential is required for the generation of Cu(I) catalyst and, thus, the click reaction.

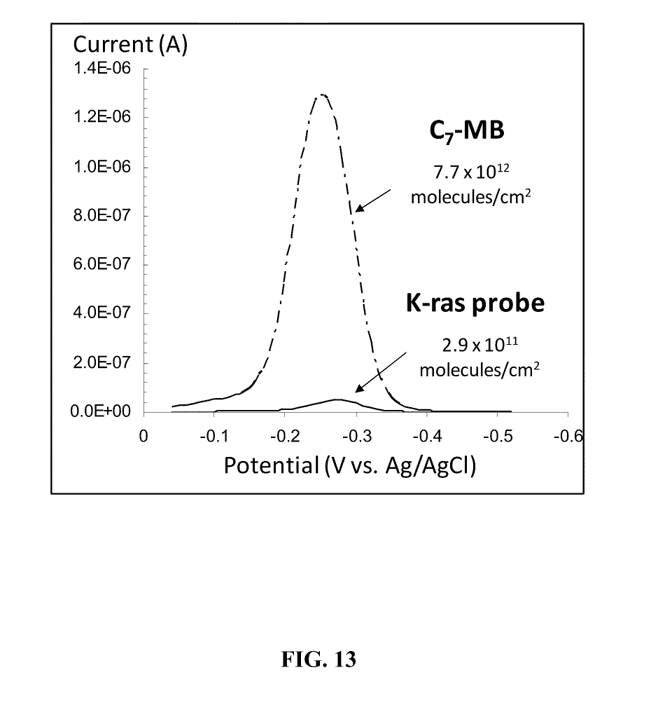

Owing to the successful results obtained with the surrogate probe, a MB-labeled DNA probe (K-ras probe) was “clicked” onto an azide-containing SAM-modified electrode and the results were compared with those obtained with the C7-MB model system. It was found that the probe density of C7-MB was significantly higher than that of the K-ras probe (FIG. 13). While there may be differences in electron transfer efficiency between the C7-MB probe and the K-ras DNA probe, it is conceivable to have significant differences in probe coverage. Specifically, this difference could be attributed to the difference in molecular sizes, where the larger DNA probe produces more steric hindrance when compared to the smaller C7-MB molecule, leading to the lower probe density (FIG. 13). Another possible reason is the difference in charges; C7-MB is singly positively charged while the K-ras probe is highly negatively charged and, thus, susceptible to electrostatic repulsions upon application of the negative click potential. Any or all of these factors may contribute to a lower “clicking” efficiency.

With the goal of increasing the DNA probe coverage, a crucial aspect for optimal sensor performance, the effect of ionic strength on probe coverage was studied. The salt (i.e., sodium, magnesium) concentration in the click mixture was optimized to ensure adequate shielding between the negatively charged phosphate backbone, thus minimizing probe-probe repulsion and probe-electrode repulsion. The results herein show that the presence of sodium and low levels of magnesium significantly increases DNA “clicking” efficiency, whereas high concentrations of magnesium impedes probe attachment (Table 1). While a low concentration of magnesium, in the presence of sodium, aids in the reduction of electrostatic repulsion between the probe and the electrode, a higher concentration of magnesium (i.e. 100 mM) presumably induces probe aggregation, thus limiting “clicking” efficiency, as indicated by the low probe density. Based on these results, the optimal salt concentration for DNA probe immobilization was 0.1 M NaCl with 2.7 mM MgCl₂, and this salt combination was used in the remaining studies.

TABLE 1
K-ras DNA probe density as a function of
the ionic strength of the click mixture.

| Salts Added to the | Probe Density |
|---|---|
| “Click” Mixture | (molecules/cm²) |
| None (deionized water) | 8.87 × 10⁹ |
| 100 mM Na only | 3.97 × 10¹¹ |
| 100 mM Na + 2.7 mM Mg | 5.16 × 10¹¹ |
| 100 mM Na + 5.9 mM Mg | 3.03 × 10¹¹ |
| 100 mM Mg only | 4.04 × 10⁸ |

The reported values are averages of two separate experiments

After having successfully optimized both the click potential and the ionic strength, the two main challenges in potential-assisted click chemistry, this approach was applied to the fabrication of a 3-pixel E-DNA sensor array. The three MB-modified DNA probes used in this study were designed to target specific cancer-related gene sequences (i.e., the Brca2, p53 and K-ras gene). To fabricate the sensor array, the C11-0H/C11-N3 SAM-modified electrode array was incubated in a click mixture containing the specific DNA probe while applying a potential of −500 mV to the desired pixel (i.e., Pixel 1) and holding the other two pixels at OPC. All three pixels were exposed to the click mixture, but only the pixel at which the potential was applied was modified with DNA probes, pixels that were held at OPC remained unaltered. Using this approach, the 3 pixels in the array were sequentially modified with their respective DNA probes prior to target interrogation. Pixel 1 was modified with the Brac2 probes, whereas Pixel 2 and 3 were modified with the p53 and K-ras probes, respectively. Sensor interrogation with the full complement target DNA was conducted one at a time to determine possible cross-reactivity (i.e., specific conjugation of DNA probes onto pixels that were held at OPC). In this study, Pixel 3 was the last to be modified with probe DNA (K-ras), but no substantial signal suppression (i.e., indication of hybridization) was evident when challenged with target DNA complementary to the other two probes, indicating minimal cross reactivity during the functionalization steps (FIG. 9, FIG. 14).

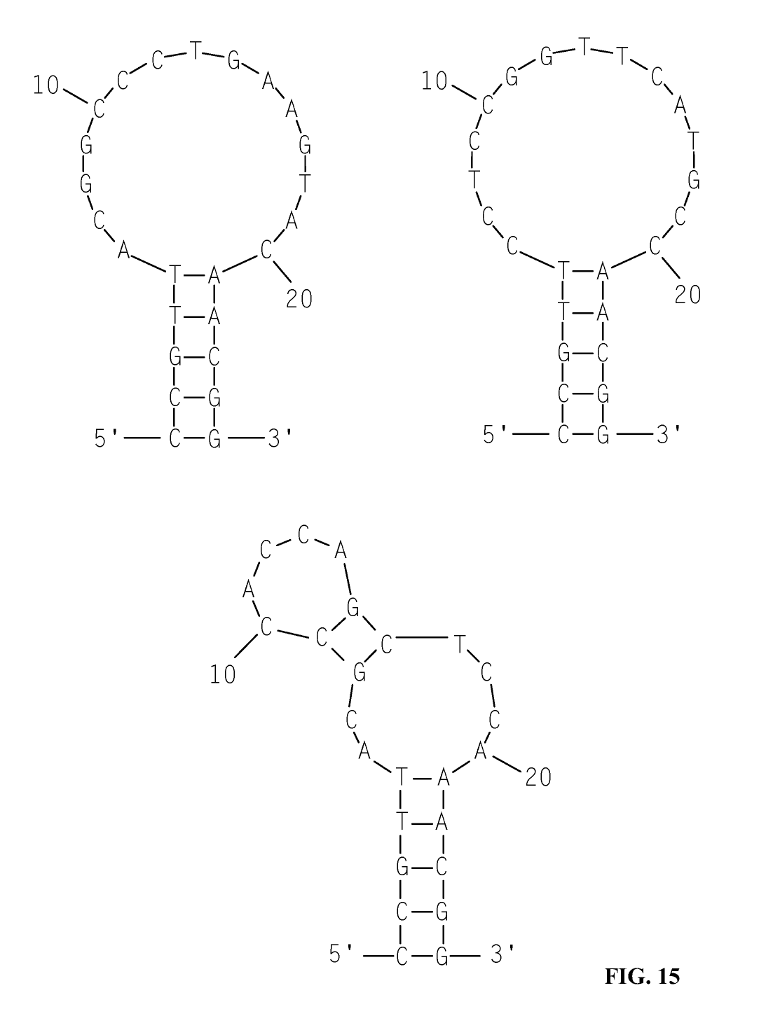

With respect to sensor performance, it was found that the sensors modified with Brca2 and p. 53 probes responded faster (saturation time˜13 min) and showed larger signal suppression (˜72%) when compared to the sensor functionalized with the K-ras probe, which showed a signal saturation time of ˜55 min and signal suppression of ˜54% (FIG. 9, FIG. 14). The faster and better sensor response observed with the Brca2 and p53 probes was probably due to the design of the probe in which both are normal stem loop probes; on the other hand, the K-ras probe had a mini-loop within the major loop region (FIG. 15). Thus, target hybridization to the Brca2 and p53 probes would be both kinetically and thermodynamically more favorable when compared to the K-ras probe. It was noted that the slower hybridization kinetics and lower signal suppression observed with the K-ras probe were evident even when the probe was immobilized on a gold disk electrode using conventional click chemistry with sodium ascorbate as the reducing agent. In addition, the Brca2 probe did not regenerate (˜84% regeneration) as well as the p53 (˜101% regeneration) and K-ras (˜106% regeneration) probes. The lack of complete sensor regeneration with the Brca2 probe could be attributed to the particular probe sequence. In addition, presumably, the observed signal recovery in excess of 100% arises because the AC current observed with the E-DNA sensors, in particular, those fabricated on thin-film gold electrodes, are rather sensitive to the force with which the washing step is conducted. Significantly, however, despite the slight differences in sensor behavior among the three probes, E-DNA sensors fabricated via potential-assisted click chemistry can be used repeatedly and have shown sensor stability of at least ˜8 hours.

In summary, the first E-DNA sensor array using potential-assisted click chemistry has been described herein. The sensors exhibit comparable attributes as sensors fabricated via the conventional click chemistry approach. Thus, the versatile sensor array fabrication approach described herein can be used in the fabrication of other folding-based electrochemical biosensors.

### Example 3

### Fabrication of Other Electrochemical Sensors Via Potential-Assisted “Click” Chemistry

Preparation of a “Click” Chemistry-Based E-AB Sensor for VEGF Detection.

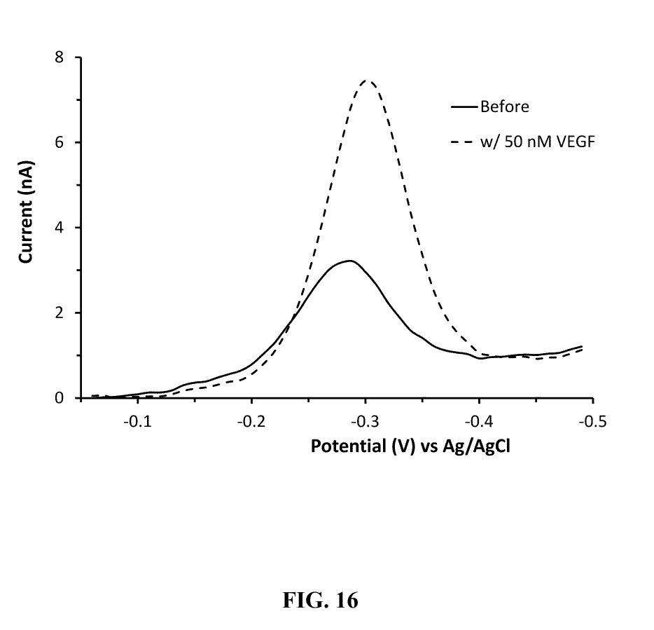

The VEGF DNA aptamer probe was modified with a C3-alkyne at the 5′-end for conjugation to the surface azide and the probe sequence was as follows: 5′ HCC≡C—(CH₂)₃-TTC CCG TCT TCC AGA CAA GAG TGC AGG G-(CH₂)₇—NH-methylene blue-3′ (SEQ ID NO:11). A cleaned gold electrode was placed in a 10 μM solution of HS-C11-N₃ prepared in EtOH for 10 min. The electrode was subsequently incubated in a 2 mM ethanolic HS-C11-OH solution for 3 hr to complete the monolayer formation. The mixed monolayer-modified electrode was then rinsed with H₂O, and transferred to a click mixture containing 1.8 mM TBTA, 800 μM sodium ascorbate, 800 μM CuSO₄ and 5 μM of alkyne-modified aptamer probe 1:1 DMSO/H₂O. The click mixture also contained 100 mM NaCl and 2.7 mM MgCl₂. The click reaction was allowed to proceed for 30 min in dark. To remove physically adsorbed probes, the electrode was rinsed thoroughly with DI water, 5% tween, EtOH and then again in the reverse order. The results using this sensor are shown in FIG. 16.

This protocol is very similar to the protocol used in the fabrication of the E-DNA sensor. The amount of salt in the click mixture is comparable to that used in the fabrication of the E-DNA sensor. The salt used in this protocol was needed to neutralize the negative charges on the aptamer probes to allow efficient conjugation of the probes to the electrode surface. The main difference in this protocol is the higher concentration of TPTA, sodium ascorbate and CuSO₄ in the click mixture.

Preparation of a “Click” Chemistry-Based E-AB Sensor for PDGF Detection.

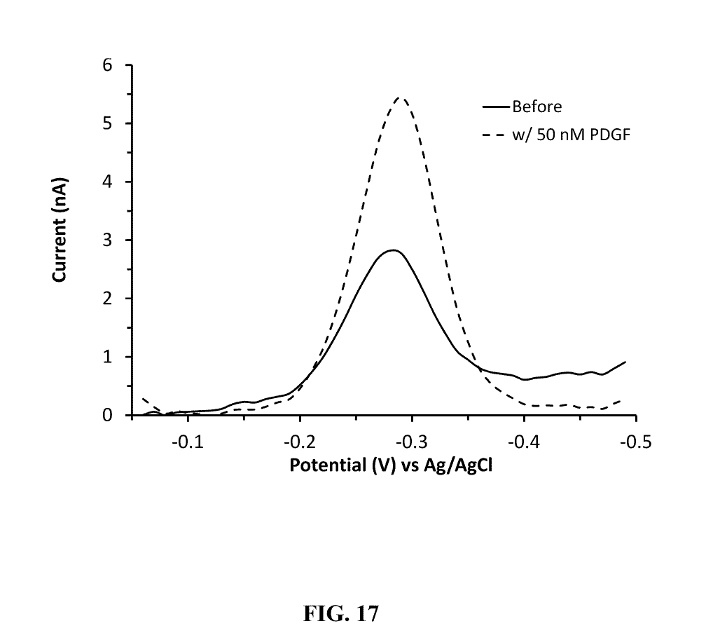

The PDGF DNA aptamer probe was modified with a C3-alkyne at the 5′-end for conjugation to the surface azide and the probe sequence was as follows: 5′ HCC≡C—(CH₂)₃-CAG GCT ACG GCA CGT AGA GCA TCA CCA TGA TCC TG-(CH₂)₇—NH-methylene blue-3′ (SEQ ID NO:12). A cleaned gold electrode was placed in a 3 μM solution of HS-C11-N₃ prepared in EtOH for 10 min. The electrode was subsequently incubated in a 2 mM ethanolic HS-C11-OH solution for 3 hr to complete the monolayer formation. The mixed monolayer-modified electrode was then rinsed with H₂O and transferred to a click mixture containing 1.8 mM TBTA, 800 μM sodium ascorbate, 800 μM CuSO₄ and 5 μM of alkyne-modified aptamer probe 1:1 DMSO/H₂O. The click mixture also contained 100 mM NaCl and 0.9 mM MgCl₂. The click reaction was allowed to proceed for 30 min in the dark. To remove physically adsorbed probes, the electrode was rinsed thoroughly with DI water, 5% tween, EtOH and then again in the reverse order. The results using this sensor are shown in FIG. 17.

The above protocol is very similar to the protocol used in the fabrication of the VEGF sensor. The concentration of NaCl in the click mixture in this protocol was comparable to that used in the fabrication of the VEGF sensor; however, the concentration of MgCl₂ was lower in this protocol (0.9 mM). Higher concentration of MgCl₂ presumably induces extensive secondary structure in the aptamer probe, which could “bury” the alkyne moiety—the key to click chemistry, thereby lowering the efficiency of probe conjugation to the electrode surface. By way of example, the use of 2.7 mM MgCl₂ in the click mixture was detrimental to the click reaction, resulting in a sensor with extremely low probe coverage (i.e., no observable methylene blue reduction signal). The effect of MgCl₂ appeared to be more pronounced for this sensor, which utilized a longer aptamer (35 bases), whereas the shorter VEGF aptamer (28 bases) has fewer constraints regarding divalent cation concentrations in the click mixture.

Preparation of a “Click” Chemistry-Based E-PB Sensor for Detection of Anti-HIV Antibodies.

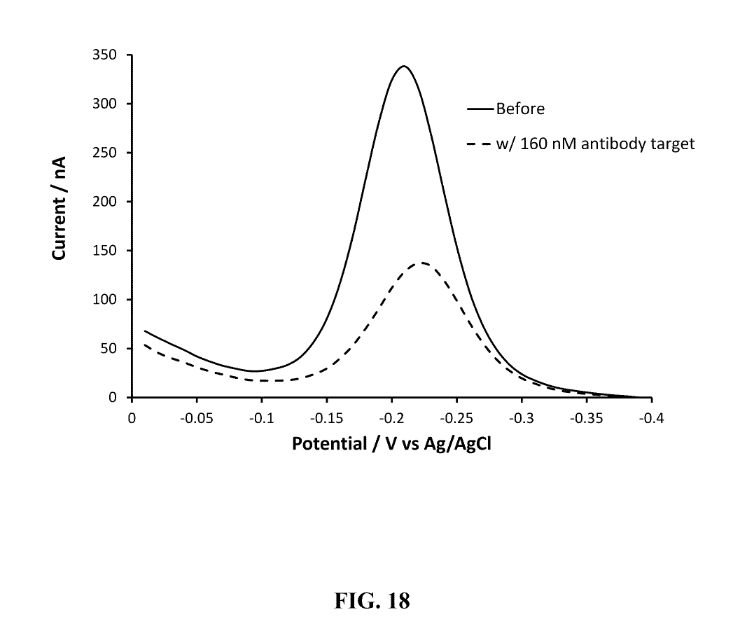

The p24 peptide probe was modified with a C3-alkyne at the amino-terminus for conjugation to the surface azide. The methylene blue label was conjugated to the peptide via an added lysine residue at the C-terminus. The peptide probe sequence was as follows: HCC≡C—(CH₂)₃-TINEEAAEWDRVHP-K-methylene blue (SEQ ID NO:13). A cleaned gold electrode was placed in a 150 μM solution of alkanethiols (HS-C11-N₃ and HS-C9-OH) prepared in EtOH for 10 min. The electrode was subsequently incubated in a 2 mM ethanolic HS-C9-OH solution for 20 hr to complete the monolayer formation. The mixed monolayer-modified electrode was then rinsed with EtOH and transferred to a click mixture containing 1.4 mM TBTA, 830 μM sodium ascorbate, 440 μM CuSO₄ and 4.3 μM of alkyne-modified peptide 1:1 DMSO/H₂O. The click mixture also contained 65 mM NaCl. The click reaction was allowed to proceed for 3 hr in the dark. To remove physically adsorbed probes, the electrode was rinsed thoroughly with DI water prior to use. The results using this sensor are shown in FIG. 18.

Overall, this protocol is similar to the protocol used in the fabrication of the E-DNA and E-AB sensors. The concentration of NaCl in the click mixture in this protocol was lower than that used in the fabrication of the DNA-based sensors (E-DNA and E-AB sensors) since peptides, without the negatively charged phosphate backbone, has a lower salt stringency. Specifically, the addition of MgCl₂ was not necessary for the fabrication of this sensor. However, peptide properties vary significantly depending on the sequence, the amount of monovalent and divalent cations required for efficient peptide probe immobilization could differ and will need to be adjusted accordingly.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An electrochemical biosensor, wherein said biosensor comprises:
at least two electrodes and a binding ligand, wherein one end of the binding ligand is linked to at least one of the electrodes via a 1,2,3 triazole moiety, wherein a second end of the binding ligand comprises a redox tag.

2. The electrochemical biosensor of claim 1, wherein the binding ligand is selected from the group consisting of nucleic acids, aptamers, polypeptides, and proteins.

3. The electrochemical biosensor of claim 1, wherein the electrochemical biosensor is disposable.

4. The electrochemical biosensor of claim 1, wherein the electrochemical biosensor is reusable.

5. The electrochemical biosensor of claim 1, wherein one or more of the electrodes are gold-plated.

6. The electrochemical biosensor of claim 1, wherein the redox tag is methylene blue.

7. A method of making an electrochemical biosensor, comprising:
providing an electrochemical sensor comprising a working electrode, a reference electrode, and a counter electrode; and
attaching a binding ligand to the substrate using click chemistry, wherein said binding ligand comprises a redox tag.

8. The method of claim 7, wherein said binding ligand is selected from the group consisting of nucleic acids, aptamers, polypeptides, and proteins.

9. The method of claim 7, wherein the electrochemical biosensor is disposable.

10. The method of claim 7, wherein the electrochemical biosensor is reusable.

11. The method of claim 7, wherein the redox tag is methylene blue.

12. A method of detecting the presence or absence of a target analyte in a sample, comprising:
contacting an electrochemical biosensor of claim 1 with a sample;
determining whether or not the electrochemical biosensor exhibits a change in redox potential;
wherein a change in the redox potential is indicative of the presence of the target analyte and wherein little to no change in the redox potential is indicative of the absence of the target analyte.

13. The method of claim 12, wherein the sample is a biological sample.

14. The method of claim 12, wherein the sample is an environmental sample.
